(19) United States
(12) Patent Application Publication
(10) Pub. No.: US 2025/0054635 A1
(43) Pub. Date: Feb. 13, 2025

(54) MACHINE LEARNING-BASED METHODS AND SYSTEMS FOR PREDICTING RUNNING-RELATED INJURIES

(71) Applicant: RunWise Al LLC, Jacksonville, FL (US)

(72) Inventor: Bjorn Gunnar Anderson, Jacksonville, FL (US)

(21) Appl. No.: 18/793,839

(22) Filed: Aug. 4, 2024

Related U.S. Application Data
(60) Provisional application No. 63/532,273, filed on Aug. 11, 2023.

(51) Int. Cl. G16H 50/30; G16H 10/60; G16H 20/30

(52) U.S. Cl. CPC G16H 50/30; G16H 10/60; G16H 20/30

(57) ABSTRACT

An example method for predicting risk of running-related injury, includes: retrieving a first dataset including running-related data; comparing a first distribution of the first dataset to a second distribution of a second dataset including running-related data; based on the comparison, selecting one of the first dataset or the second dataset; scaling a runner profile from the first dataset based on the selected one of the first dataset or the second dataset; inputting, into a trained machine learning model, the runner profile; and predicting, using the trained machine learning model, a risk of a musculoskeletal injury based on the runner profile.

### CROSS REFERENCE TO RELATED APPLICATIONS

This application claims priority to U.S. provisional application No. 63/532,273, filed Aug. 11, 2023, titled “MACHINE LEARNING-BASED METHODS AND SYSTEMS FOR PREDICTING RUNNING-RELATED INJURIES,” the disclosure of which is incorporated herein by reference in its entirety.

### BACKGROUND

Running is a popular activity. For example, in the United States, millions of people maintain fitness by running on a regular basis. Running, however, poses a high risk of injury due to the repetitive stress on the runner's body. By some estimates, more than 50 percent of runners experience an injury each year. During time off, runners lose fitness, miss opportunities, and experience adverse physical and mental health effects. Unfortunately, preventing injuries is an extremely difficult task. In fact, conventional injury preventive measures are often either subjective (e.g., listen to your body) or rules of thumb (e.g., avoid a week-to-week mileage increase of greater than 10%). These conventional prevention methods are also inaccurate. Moreover, researchers have not yet uncovered any predictive characteristics (e.g., strength, flexibility, biomechanics, injury history, etc.) to identify which runners are likely to get injured and/or why so. See Hutchinson, Alex, The Elusive Art of Predicting Injuries, Outside Online.com, published May 7, 2021, https://www.outsideonline.com/2423442/running-injuries-prediction-research (accessed May 8, 2021). There is therefore a need in the art for tools to predict running-related injuries.

### SUMMARY

In some aspects, the techniques described herein relate to a method for predicting risk of running-related injury, including: retrieving a first dataset including running-related data; comparing a first distribution of the first dataset to a second distribution of a second dataset including running-related data; based on the comparison, selecting one of the first dataset or the second dataset; scaling a runner profile from the first dataset based on the selected one of the first dataset or the second dataset; inputting, into a trained machine learning model, the runner profile; and predicting, using the trained machine learning model, a risk of a musculoskeletal injury based on the runner profile.

In some aspects, the step of comparing the first distribution of the first dataset to the second distribution of the second dataset includes using a statistical technique. In some aspects, the statistical technique optionally includes: calculating respective summary statistics for each of the first dataset and the second dataset; and comparing the respective summary statistics of the first dataset to the respective summary statistics of the second dataset. In some aspects, the statistical technique optionally includes a statistical test that quantifies a similarity of the first dataset and the second dataset.

In some aspects, the step of comparing the first distribution of the first dataset to the second distribution of the second dataset includes using a visualization technique. In some aspects, the visualization technique optionally includes: creating respective histograms for each of the first dataset and the second dataset; plotting the respective histograms for each of the first dataset and the second dataset; and comparing the respective histogram for the first dataset to the respective histogram for the second dataset.

In some aspects, the step of scaling the runner profile from the first dataset based on the selected one of the first dataset or the second dataset includes standardizing the runner profile from the first dataset based on at least one characteristic of the selected one of the first dataset or the second dataset.

In some aspects, the step of scaling the runner profile from the first dataset based on the selected one of the first dataset or the second dataset includes normalizing the runner profile from the first dataset based on at least one characteristic of the selected one of the first dataset or the second dataset.

In some aspects, the first dataset is an inference dataset, and the second dataset is a training dataset. In some aspects, the selected one of the first dataset or the second dataset is the inference dataset.

In some aspects, the first dataset and the second dataset include running-related data for a same runner.

In some aspects, each of the first dataset and the second dataset includes running-related data for a different runner.

In some aspects, the runner profile includes at least one volume metric, at least one intensity metric, and at least one long run fraction metric. In some aspects, the at least one volume metric includes one or more of a short-term volume metric, a medium-term volume metric, and a long-term volume metric. In some aspects, the at least one intensity metric includes one or more of a short-term intensity metric, a medium-term intensity metric, and a long-term intensity metric. In some aspects, the at least one long run fraction metric includes one or more of a short-term long run fraction metric, a medium-term long run fraction metric, and a long-term long run fraction metric. In some aspects, the runner profile further includes one or more of at least one consistency metric, at least one variability metric, at least one dynamic metric, or at least one physiological metric.

In some aspects, the trained machine learning model is configured to predict the risk of the musculoskeletal injury by classifying the runner profile into one of a plurality of risk categories.

In some aspects, the trained machine learning model is configured to predict the risk of the musculoskeletal injury by providing a probability of the musculoskeletal injury.

In some aspects, the trained machine learning model is a deep learning model.

In some aspects, the trained machine learning model is an artificial neural network.

In some aspects, the method further includes adjusting a training plan based on the predicted risk of the musculoskeletal injury.

In some aspects, the techniques described herein relate to a system for predicting risk of running-related injury, including: at least one processor and at least one memory, the at least one memory having computer-executable instructions stored thereon that, when executed by the at least one processor, cause the at least one processor to: receive a first dataset including running-related data; compare a first distribution of the first dataset to a second distribution of a second dataset including running-related data; based on the comparison, select one of the first dataset or the second dataset; scale a runner profile from the first dataset based on the selected one of the first dataset or the second dataset; input, into a trained machine learning model, the runner profile; and predict, using the trained machine learning model, a risk of a musculoskeletal injury based on the runner profile.

In some aspects, the techniques described herein relate to a method for predicting risk of running-related injury, including: retrieving an inference dataset including running-related data, where the running-related data includes a plurality of metrics for each of a plurality of training periods; scaling a runner profile from the inference dataset based on at least one characteristic of the inference dataset, where the runner profile includes the plurality of metrics for a single training period; inputting, into a trained machine learning model, the runner profile; and predicting, using the trained machine learning model, a risk of a musculoskeletal injury based on the runner profile.

In some aspects, the trained machine learning model is trained using a training dataset, where the training dataset is different than the inference dataset. In some aspects, the inference dataset and the training dataset include running-related data for a same runner. In some aspects, each of the inference dataset and the training dataset includes running-related data for a different runner.

In some aspects, the at least one characteristic of the inference dataset includes a mean or a standard deviation.

In some aspects, the plurality of metrics include at least one volume metric, at least one intensity metric, and at least one long run fraction metric.

In some aspects, the method further includes adjusting a training plan based on the predicted risk of the musculoskeletal injury.

In some aspects, the techniques described herein relate to a system for predicting risk of running-related injury, including: at least one processor and at least one memory, the at least one memory having computer-executable instructions stored thereon that, when executed by the at least one processor, cause the at least one processor to: receive an inference dataset including running-related data, where the running-related data includes a plurality of metrics for each of a plurality of training periods; scale a runner profile from the inference dataset based on at least one characteristic of the inference dataset, where the runner profile comprises the plurality of metrics for a single training period; input, into a trained machine learning model, the runner profile; and predict, using the trained machine learning model, a risk of a musculoskeletal injury based on the runner profile.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating a machine learning model operating in inference mode according to an implementation described herein.

FIG. 2 is a table illustrating an example running-related dataset according to an implementation described herein.

FIG. 3A is a flowchart illustrating example operations for predicting risk of running-related injury according to an implementation described herein. FIG. 3B is a flowchart illustrating example operations for predicting risk of running-related injury according to another implementation described herein.

FIGS. 4A-4D are tables illustrating the mean and standard deviation of example inference and training datasets according to an implementation described herein. FIGS. 4A and 4B are tables illustrating the mean and standard deviation of an example inference dataset, respectively. The example inference dataset includes 40 weeks (i.e. between the week of Oct. 31, 2022 and the week of Jul. 31, 2023) of running-related data, which is grouped by week, for an example runner (the present inventor). FIGS. 4C and 4D are tables illustrating the mean and standard deviation of an example training dataset, respectively. The example training dataset includes more than 5 years (i.e., between about Jan. 1, 2018 and Apr. 17, 2023) of running-related data, which is grouped by week, for the example runner (the present inventor).

FIG. 5 is an example computing device.

FIG. 6 is a table illustrating example feedforward artificial neural network (ANN) architecture and hyperparameters according to an example described herein. The example ANN was trained using a scaled, augmented dataset. The ANN has 12 input nodes for receiving short-, medium-, and long-term metrics for each of volume, intensity, consistency, and long run fraction (i.e., the model “features”).

FIG. 7 is a graph illustrating AUC for the ANN of FIG. 6 during training.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately” when referring to a measurable value such as an amount, a percentage, and the like, is meant to encompass variations of +20%, +10%, +5%, or +1% from the measurable value.

Described herein are machine learning-based systems and methods for predicting risk of musculoskeletal injury in a runner. As noted above, runners are at high risk of injury due, at least in part, to the repetitive stress running imposes on the human body. For example, more than 50% of runners (˜80% according to some estimates) experience an injury each year. This is particularly true for long distance runners. The machine learning-based systems and methods described herein can predict risk of musculoskeletal injury based on patterns present in running-related data. For example, the interrelationship between running volume, intensity, consistency, variability, fractional contribution of long run, and other characteristics is highly complex. Machine learning is a technical tool that is capable of analyzing complex data and identifying patterns in data. As described herein, the machine learning-based systems and methods analyze the interrelationship between various metrics present in a runner's data.

The present disclosure is concerned with generalization, i.e., a trained model's ability to perform well on new, unseen data that it has not encountered during its training phase. As used herein, new data or unseen data refers to data not used to train a machine learning model. In other words, the new data or unseen data is not included in the training dataset. A model that generalizes well is able to learn the underlying patterns from the training data and apply those patterns to make accurate predictions or classifications on data it has not seen before such as new or unseen inference data. Thus, an objective of machine learning is not just to perform well on the training data but to produce accurate predictions on unseen data as well. Generalization is a measure of how well a model has learned the relevant features and patterns from the training data without memorizing the data itself.

Data preprocessing is one aspect that can impact the trained model's ability to generalize to unseen data. And as described below, the datasets of the present disclosure-running-related datasets-pose challenges for the trained model's ability to generalize. Thus, the present disclosure includes steps to ensure better preprocessing in order to improve the trained model's ability to generalize. As described herein, the model is trained on using a training dataset comprising running-related data, and it is then deployed to make predictions on inference data. The inference data is unseen data (i.e., it was not used for training the model). In some implementations, the training and inference datasets include running-related data for a same runner. In these implementations, the runner's inference data may cover a different period of time than data included in the training data such that the quantity and/or quality of the runner's inference data (e.g., including consistency, volume, intensity, long run fraction, etc. metrics) is different than the quantity and/or quality of the runner's training data (e.g., including consistency, volume, intensity, long run fraction, etc. metrics). For example, the inference data may include the runner's data for a higher volume and/or intensity training period (e.g. marathon training cycle), while the training data (which was used to train the model) includes lower volume and/or intensity periods. In this case, the distributions of inference data and training data are expected to be different. Alternatively, in other implementations, the training and inference datasets include running-related data for a different runner. In these implementations, the quantity and/or quality of the inference data (e.g., including consistency, volume, intensity, long run fraction, etc. metrics) for one runner will be different than the quantity and/or quality of the training data for a different runner (e.g., including consistency, volume, intensity, long run fraction, etc. metrics). Again, in this case, the distributions of inference and training data are expected to be different.

It is generally good practice in machine learning to scale the inference data in the same way as the training data. This may not always be the best practice for datasets of the present disclosure. For example, there is a chance that the training data is not representative of the inference data in the present disclosure due to the type of data (i.e., running-related data). The trained model of the present disclosure is configured to predict a running-related injury, which can be considered an anomaly (e.g., a data point that is significantly different from the normal data). When anomaly detection is the goal, it may be difficult to obtain a representative set of training data that includes a sufficient number of anomalous examples. In such cases, the inference data can instead be scaled based on itself (e.g., using the mean and standard deviation of the inference data). The present disclosure therefore includes steps for analyzing respective distributions of the training and inference datasets. Based on this analysis, the inference data is scaled accordingly. Importantly, in cases where distributions are significantly different, inference data is scaled based on its own characteristics (e.g., mean and/or standard deviation). Thus, the machine learning-based systems and methods described herein provide improvements over existing technologies by performing a data analysis on the training and inference datasets and then not always scaling inference data in the same manner as training data as would typically be done. This improves the trained machine learning model's ability to generalize to new or unseen data.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

As used herein, musculoskeletal injuries affect a runner's bones, joints, or soft tissues such as muscles, tendons, ligaments, or other connective tissue. Running-related injuries include, but are not limited to, those affecting the feet, knees, upper or lower legs, hips, pelvis, or groin. Example running-related musculoskeletal injuries include, but are not limited to, stress fractures, tendonitis, plantar fasciitis, iliotibial (IT) band syndrome, strains, and sprains. Additionally, this disclosure contemplates that a musculoskeletal injury forces a runner to rest (not run) for an extended period of time (e.g., from 3-5 days or longer such as several weeks, months, or even longer). Thus, as used herein, a running-related injury results in a runner taking 3 or more consecutive days of rest. Optionally, a running-related injury results in a runner taking at least 5 consecutive days of rest.

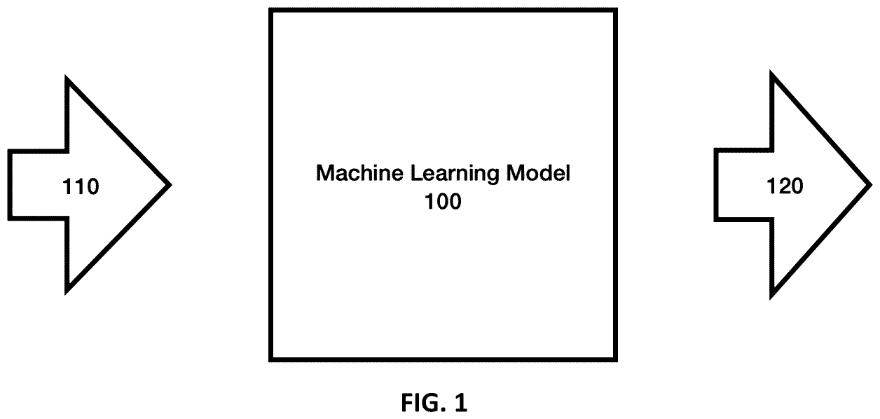

Referring now to FIG. 1, a block diagram illustrating a machine learning model 100 is shown. In FIG. 1, the machine learning model 100 is operating in inference mode. In other words, the machine learning model 100 has already been trained with a data set (or “dataset”). Techniques for training a machine learning model for predicting running-related injuries are described in U.S. Pat. No. 11,515,045 to Anderson, titled “Predicting risk of running-related injury using a machine learning model and related machine learning training methods,” the disclosure of which is incorporated herein by reference in its entirety. This disclosure contemplates that the machine learning model 100 is a supervised learning model. According to supervised learning, the machine learning model 100 “learns” a function that maps an input 110 (sometimes referred to herein as the “features”) to an output 120 (sometimes referred to herein as the “target”) based on a data set, which includes a plurality of samples from a running-related training dataset tagged with one or more labels (e.g., the injury/no injury tags described herein), during model training mode. It should be understood that supervised learning is provided only as an example. This disclosure contemplates that the machine learning model 100 may be a semi-supervised learning model in some implementations. Semi-supervised learning models are trained with a data set including both labeled data as well as unlabeled data.

The machine learning model 100 shown in FIG. 1 can be an artificial neural network. Optionally, the machine learning model 100 is a deep neural network, which includes multiple hidden layers between the input and output layers (described below). An artificial neural network is a computing system including a plurality of interconnected neurons (e.g., also referred to as “nodes”). This disclosure contemplates that the nodes can be implemented using a computing device (e.g., a processing unit and memory as described herein). The nodes can optionally be arranged in a plurality of layers such as input layer, output layer, and one or more hidden layers. Each node is connected to one or more other nodes in the artificial neural network. For example, each layer has a plurality of nodes, where each node is connected to all nodes in the previous layer. The nodes in a given layer are not interconnected with one another, i.e., the nodes in a given layer function independently of one another. As used herein, nodes in the input layer receive data (sometimes referred to herein as the “features” or input 110) from outside of the artificial neural network, nodes in the hidden layer(s) modify the data between the input and output layers, and nodes in the output layer provide the results (sometimes referred to herein as the “target” or output 120).

Each node in the artificial neural network is configured to receive an input and implement a function (sometimes referred to herein as the “activation function”). In other words, the activation function defines the node output for a given input. Activation functions include, but are not limited to, binary step, sigmoid, tanh, and rectified linear unit (ReLU). Additionally, each node is associated with a respective weight. Artificial neural networks are trained with a data set to minimize or maximize an objective function, which is a measure of the artificial neural network's performance. The objective function may be a cost function. Cost functions include, but are not limited to, mean squared error (MSE), mean absolute error, L1 loss (least absolute deviations), L2 loss (least squares loss), and cross-entropy loss. Training algorithms for artificial neural networks include, but are not limited to, backpropagation (BP). The training algorithm tunes the node weights and/or bias to minimize or maximize the objective function. For example, BP involves computing the gradient of the objective function with respect to the respective weights for each of the nodes. It should be understood that any algorithm that finds the minimum or maximum of the objective function can be used to for training an artificial neural network. Although artificial neural networks are provided as an example, this disclosure contemplates that the machine learning model 100 can be other types of models including, but not limited to, a logistic regression model or a support vector machine.

As described above, the machine learning model 100 is trained to map the input 110 to the output 120. In the examples described herein, the input 110 is a runner profile, and the output 120 is a risk of musculoskeletal injury, e.g., running-related musculoskeletal injury. As used herein, the risk of musculoskeletal injury can be a classification (e.g., injury or no injury) in some implementations or a predicted risk value (e.g., regression) in other implementations. As described above, musculoskeletal injuries affect a runner's bones, joints, or soft tissues and also force the runner to rest for an extended time period. The runner profile includes one or more “features” that are input into the machine learning model 100, which predicts risk of musculoskeletal injury based on the features. The risk of musculoskeletal injury is therefore the “target” of the machine learning model 100.

This disclosure contemplates that the running-related datasets described herein can be obtained from a runner's log, e.g., the record used to track running-related information such as mileage, running duration, physiological data, environmental conditions, injuries, or other information related to running. Optionally, the runner's log is maintained in an electronic medium. For example, Internet-based services for tracking fitness data are in common use by runners. Example Internet-based services include, but are not limited to, the STRAVA mobile app and website of Strava, Inc. of San Francisco, California and GARMIN CONNECT mobile app and website of Garmin International of Olathe, Kansas. It should be understood that the STRAVA and GARMIN CONNECT mobile apps and websites are provided only as example Internet-based services. This disclosure contemplates that other electronic and/or Internet-based services may be used to track running-related data.

Internet-based services maintain a vast amount of running-related data for a plurality of runners. For example, the STRAVA mobile app and website had approximately 76 million users in 2021. Running-related data includes, but is not limited to, global positioning system (GPS) route data (e.g., XML format files such as GPX or TCX files); mileage; duration; pace; speed; sensor data (e.g., heart rate monitor, accelerometer, etc.); dynamic data (e.g., cadence, stride length); perceived effort; and free-form comments. Such running-related data is primarily measured using a device, for example, a running watch, fitness tracker, or mobile phone. These devices include built-in location service such as GPS and, optionally, built-in or external sensors. An example running watch is the GARMIN FORERUNNER watch of Garmin International of Olathe, Kansas. It should be understood that the GARMIN FORERUNNER watch is provided only as an example. This disclosure contemplates that other devices may be used to measure running-related data. Alternatively or additionally, running-related data may be entered or altered by the runner.

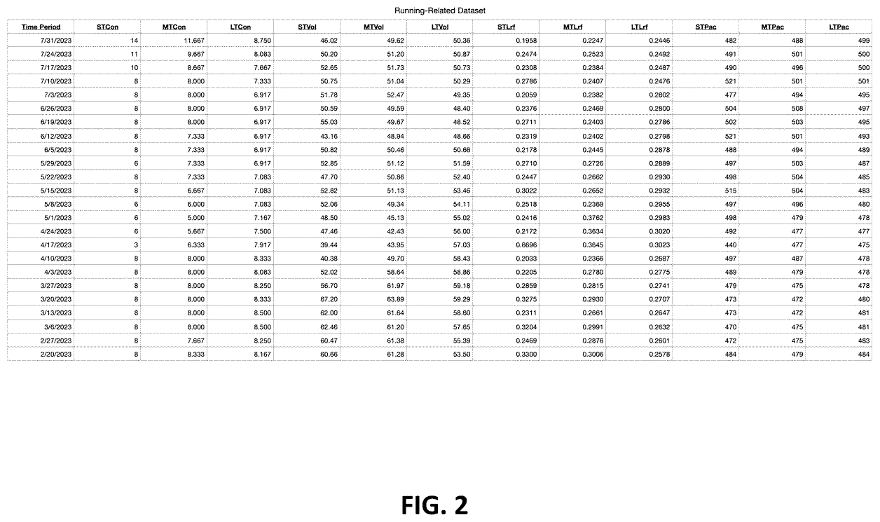

Referring now to FIG. 2, an example dataset comprising running-related data is shown. The running data was obtained from an electronic runner's log, and metrics (e.g., volume, intensity, long run fraction, consistency) were calculated as described below using the Python programming language. The example dataset shown in FIG. 2 includes 24 rows of running-related data, which is grouped by week. Accordingly, each row corresponds to a week between Feb. 20, 2023 and Jul. 31, 2023, and each column corresponds to a metric. It should be understood that the number of weeks of data in FIG. 2 (i.e., 24 weeks) is provided only as an example. In some implementations, the dataset may include more than 24 weeks of running-related data, e.g., 25, 26, 27, 28, 29, 30, 31, 32, 33, 34, 35, 36, or more weeks of running-related data. Optionally, the dataset may include 36 weeks of running-related data. Optionally, the dataset may include 52 weeks of running-related data. Alternatively, the dataset may include less than 24 weeks of running-related data, e.g., 23, 22, 21, 20, 19, 18, or less weeks of running-related data. Optionally, the dataset may include 18 weeks of running-related data. It should also be understood that the running-related data may include more or less metrics than shown in FIG. 2.

As described below, metrics are provided for short-term, medium-term, and long-term periods. As used herein, a short-term period represents a training period. A training period can optionally be a 7 day period (e.g., a calendar week). It should be understood that a training period may be more or less than 7 days (e.g., a 10-day or 5-day period). It should also be understood that the training period length can be selected by a runner. As used herein, a medium-term period includes a plurality of training periods. The number of training periods in a medium-term period is selected to create metrics representing the transient fitness level of and stress on the runner. For example, the medium-term period can be a 2-4 week period (i.e., 2-4, 7-day training periods). Optionally, the medium-term period can be a 3 week period (i.e., three, 7-day training periods). It should be understood that 2-4 weeks is only provided as an example. As used herein, a long-term period includes a plurality of training periods, which is greater than the number of training periods of the medium-term period. The number of training periods in a long-term period is selected to create metrics representing the base fitness level of and stress on the runner. For example, the long-term period can be a 10-14 week period (i.e., 10-14, 7-day training periods). Optionally, the long-term period can be a 12 week period (i.e., twelve, 7-day training periods). It should be understood that 10-14 weeks is only provided as an example.

The running-related data includes at least one volume metric. Volume metrics include, but are not limited to, a daily volume metric, a short-term volume metric, a medium-term volume metric, and a long-term volume metric. Optionally, in some implementations as shown in FIG. 2, the volume metrics includes a short-term volume metric (STVol in FIG. 2), a medium-term volume metric (MTVol in FIG. 2), and a long-term volume metric (LTVol in FIG. 2). This disclosure contemplates that a volume metric is a measure of running time or duration (e.g., hours, minutes, seconds) and/or running distance (e.g., miles, kilometers). In FIG. 2, volume is a distance (miles). Additionally, this disclosure contemplates that the volume metrics can be obtained (e.g., received, downloaded, etc.) and/or derived from the electronic runner's log described above. As used herein, a daily volume metric is a 1-day cumulative run length (e.g., daily total), which can optionally include one or more runs. As used herein, a short-term volume metric is the cumulative run length during a training period. Additionally, as described above, a training period can optionally be a 7 day period (e.g., a calendar week). It should be understood that a training period may be more or less than 7 days (e.g., a 10-day or 5-day period). As used herein, a medium-term volume metric is an average cumulative run length over a plurality of training periods, for example, the average training period (e.g., weekly) run length over a 2-4 week period. It should be understood that 2-4 weeks is only provided as an example medium-term period. As used herein, a long-term volume metric is an average cumulative run length over a plurality of training periods, for example, the average training period (e.g., weekly) run length over a 10-14 week period. It should be understood that 10-14 weeks is only provided as an example long-term period. The short-, medium-, and long-term volume metrics represent cumulative run lengths over progressively longer periods of time. Additionally, as described above, run length can be measured by a duration and/or a distance.

The table in FIG. 2 illustrates short-term, medium-term, and long-term volume metrics. For example, the table includes short-term volume metrics (“STVol”), medium-term volume metrics (“MTVol”), and long-term volume metrics (“LTVol”) for an example runner during twenty four (24) consecutive weeks in 2023 (i.e., weeks of February 20th through July 31^{st}). In FIG. 2, the short-term, medium-term, and long-term periods are 1, 3, and 12 weeks, respectively. As described above, these lengths are provided only as examples. Additionally, it should be understood that the metrics shown in FIG. 2 were calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

The running-related data also includes at least one intensity metric. Intensity metrics include, but are not limited to, a daily intensity metric, a short-term intensity metric, a medium-term intensity metric, and a long-term intensity metric. Optionally, in some implementations as shown in FIG. 2, the intensity metrics includes a short-term intensity metric (STPac in FIG. 2), a medium-term intensity metric (MTPac in FIG. 2), and a long-term intensity metric (LTPac in FIG. 2). This disclosure contemplates that an intensity metric is a running pace or running speed. Pace is measured as a time per distance unit (e.g., minutes per mile or minutes per kilometer). Speed is measured as distance per unit time (e.g., miles per hour or kilometers per hour). In FIG. 2, intensity is a pace (seconds per mile). Additionally, this disclosure contemplates that the intensity metrics can be obtained (e.g., received, downloaded, etc.) and/or derived from the electronic runner's log described above. As used herein, a daily intensity metric is a 1-day average intensity (e.g., pace or speed). As used herein, a short-term intensity metric is the average intensity (e.g., pace or speed) during a training period. As used herein, a medium-term intensity metric is the average intensity (e.g., pace or speed) over a plurality of training periods. As used herein, a long-term intensity metric is the average intensity (e.g., pace or speed) over a plurality of training periods. The daily, short-, medium-, and long-term intensity metrics represent average intensity over progressively longer periods of time. Additionally, as described above, run intensity can be measured by pace or speed.

The table in FIG. 2 illustrates short-term, medium-term, and long-term intensity metrics. For example, the table includes short-term volume metrics (“STPac”), medium-term volume metrics (“MTPac”), and long-term volume metrics (“LTPac”) for an example runner during twenty four (24) consecutive weeks in 2023 (i.e., weeks of February 20th through July 31^{st}). In FIG. 2, the short-term, medium-term, and long-term periods are 1, 3, and 12 weeks, respectively. As described above, these lengths are provided only as examples. Additionally, it should be understood that the metrics shown in FIG. 2 were calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

The running-related data also includes at least one long run fraction metric. Long run fraction metrics include, but are not limited to, one or more of a short-term long run fraction metric (STLrf in FIG. 2), a medium-term long run fraction metric (MTLrf in FIG. 2), and a long-term long run fraction metric (LTLrf in FIG. 2). In FIG. 2, the long run fraction metric represents a long run volume (miles) divided by a training period volume (miles). For example, if a runner's longest run during a 7-day training period is 10 miles and the runner's total mileage during the 7-day training period is 50 miles, the long run fraction metric is 0.2. Additionally, this disclosure contemplates that the long run fraction metrics can be obtained (e.g., received, downloaded, etc.) and/or derived from the electronic runner's log described above. As used herein, a short-term long run fraction metric is a long run volume divided by total volume during a training period. For example, the short-term long run fraction metric is 0.2 when a runner's longest run is 10 miles during 7-day training period where total mileage is 50 miles. As used herein, a medium-term long run fraction metric is the average long run fraction metric over a plurality of training periods. For example, if a runner's long run fraction is 0.2, 0.3, and 0.4 during each of three consecutive 7-day training periods, respectively, then the medium-term long run fraction metric is 0.3. As used herein, a long-term long run fraction metric is the average long run fraction metric over a plurality of training periods. For example, if a runner's long run fraction is 0.2, 0.3, 0.4, 0.25, 0.3, 0.25, 0.2, 0.2, 0.4, 0.3, 0.25, and 0.2 during each of twelve consecutive 7-day training periods, respectively, then the long-term long run fraction metric is 0.27. The short-, medium-, and long-term long run fraction metrics capture the training period-to-training period fractional contribution of a runner's longest run to total volume over progressively longer periods of time. This disclosure contemplates that patterns predictive of injury risk are present in the volume, intensity, and long run fraction metrics found in running-related data.

The table in FIG. 2 illustrates short-term, medium-term, and long-term long run fraction metrics. For example, the table includes short-term volume metrics (“STLrf”), medium-term volume metrics (“MTLrf”), and long-term volume metrics (“LTLrf”) for an example runner during twenty four (24) consecutive weeks in 2023 (i.e., weeks of February 20th through July 31^{st}). In FIG. 2, the short-term, medium-term, and long-term periods are 1, 3, and 12 weeks, respectively. As described above, these lengths are provided only as examples. Additionally, it should be understood that the metrics shown in FIG. 2 were calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

Optionally, the running-related data also includes at least one consistency metric. Consistency metrics include, but are not limited to, a short-term consistency metric (STCon in FIG. 2), a medium-term consistency metric (MTCon in FIG. 2), and a long-term consistency metric (LTCon in FIG. 2). This disclosure contemplates that a consistency metric represents a number of running days (or number of runs) during the short-term, medium-term, and/or long-term periods. In FIG. 2, consistency is a raw number of runs during a training period. Additionally, this disclosure contemplates that the consistency metrics can be obtained (e.g., received, downloaded, etc.) and/or derived from the electronic runner's log described above. As used herein, a short-term consistency metric is the number of running days or raw number of runs during a training period. For example, if a runner ran every day Monday through Friday during a 7-day training period, then the short-term consistency metric is 5. As used herein, a medium-term consistency metric is the average consistency over a plurality of training periods. For example, if a runner ran 5, 6, and 7 days during each of three consecutive 7-day training periods, respectively, then the medium-term consistency metric is 6. As used herein, a long-term intensity metric is the average consistency over a plurality of training periods. For example, if a runner ran 5, 6, 7, 0, 1, 1, 5, 6, 7, 4, 2, and 4 days during each of twelve consecutive 7-day training periods, respectively, then the long-term consistency metric is 4. The short-, medium-, and long-term consistency metrics represent a runner's training period-to-training period consistency over progressively longer periods of time. Additionally, as described above, consistency can be measured by a number of running days or raw number of runs. This disclosure contemplates that patterns predictive of injury risk are present in the volume, intensity, long run fraction, and consistency metrics found in running-related data.

The table in FIG. 2 illustrates short-term, medium-term, and long-term consistency metrics. For example, the table includes short-term volume metrics (“STCon”), medium-term volume metrics (“MTCon”), and long-term volume metrics (“LTCon”) for an example runner during twenty four (24) consecutive weeks in 2023 (i.e., weeks of February 20th through July 31^{st}). In FIG. 2, the short-term, medium-term, and long-term periods are 1, 3, and 12 weeks, respectively. As described above, these lengths are provided only as examples. Additionally, it should be understood that the metrics shown in FIG. 2 were calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

Alternatively or additionally, the running-related data optionally includes at least one variability metric. Variability metrics include, but are not limited to, a short-term variability metric, a medium-term variability metric, and a long-term variability metric. This disclosure contemplates that a variability metric represents a number of high-intensity running days (or number of high-intensity runs) during the short-term, medium-term, and/or long-term periods. As used herein, a high-intensity run is a run requiring greater than ordinary effort by a runner. For example, a workout and a race are considered high-intensity runs. Additionally, this disclosure contemplates that the variability metrics can be obtained (e.g., received, downloaded, etc.) and/or derived from the electronic runner's log described above. As used herein, a short-term variability metric is the number of high-intensity running days or raw number of high-intensity runs during a training period. For example, if a runner ran with high-intensity (e.g., workout, race, etc.) twice during a 7-day training period, then the short-term variability metric is 2. As used herein, a medium-term variability metric is the average variability over a plurality of training periods. For example, if a runner ran with high intensity (e.g., workout, race, etc.) 2, 1, and 1 days during each of three consecutive 7-day training periods, respectively, then the medium-term variability metric is 1.33. As used herein, a long-term variability metric is the average variability over a plurality of training periods. For example, if a runner ran with high intensity (e.g., workout, race, etc.) 2, 1, 1, 0, 0, 1, 1, 2, 2, 0, 1, and 1 days during each of twelve consecutive 7-day training periods, respectively, then the long-term variability metric is 1. The short-, medium-, and long-term variability metrics capture training period-to-training period high-intensity efforts over progressively longer periods of time. Additionally, as described above, variability can be measured by a number of high-intensity running days or raw number of high-intensity runs. This disclosure contemplates that patterns predictive of injury risk are present in the volume, intensity, long run fraction, consistency, and variability metrics or combinations thereof found in running-related data.

Alternatively or additionally, the running-related data optionally includes at least one dynamic metric. Dynamic metrics include, but are not limited to, a daily dynamic metric, a short-term dynamic metric, a medium-term dynamic metric, and a long-term dynamic metric. Optionally, in some implementations, the dynamic metrics includes a short-term dynamic metric, a medium-term dynamic metric, and a long-term dynamic metric. This disclosure contemplates that a dynamic metric defines an aspect of a runner's motion. Dynamic metrics can be derived from sensor data such as accelerometer or internal sensor data. Example dynamic metrics include, but are not limited to, cadence or stride length. Additionally, this disclosure contemplates that the dynamic metrics can be obtained (e.g., received, downloaded, etc.) and/or derived from the electronic runner's log described above. As used herein, a daily dynamic metric is a 1-day average dynamic metric such as cadence or stride length. As used herein, a short-term dynamic metric is the average dynamic metric such as cadence or stride length during a training period. As used herein, a medium-term dynamic metric is the average dynamic metric such as cadence or stride length over a plurality of training periods. As used herein, a long-term dynamic metric is the average dynamic metric such as cadence or stride length over a plurality of training periods. The daily, short-, medium-, and long-term dynamic metrics represent average dynamic metrics such as cadence or stride length over progressively longer periods of time. This disclosure contemplates that patterns predictive of injury risk are present in the combination of volume, intensity, long run fraction, consistency, variability, and dynamic metrics or combinations thereof found in running-related data.

Alternatively or additionally, the running-related data optionally includes a physiological metric. Physiological metrics include, but are not limited to, a daily physiological metric, a short-term physiological metric, a medium-term physiological metric, and a long-term physiological metric. Optionally, in some implementations, the physiological metrics includes a short-term physiological metric, a medium-term physiological metric, and a long-term physiological metric. Physiological metrics include, but are not limited to, heart rate data, oxygen saturation data, or VO₂ max data, for example. Optionally, the heart rate data is average heart rate or heart rate variability (HRV). Additionally, this disclosure contemplates that the physiological metrics can be obtained (e.g., received, downloaded, etc.) and/or derived from the electronic runner's log described above. As used herein, a daily physiological metric is a 1-day average physiological metric such as heart rate during a run. As used herein, a short-term physiological metric is the average physiological metric such as running heart rate during a training period. As used herein, a medium-term physiological metric is the average physiological metric such as running heart rate over a plurality of training periods. As used herein, a long-term physiological metric is the average physiological metric such as running heart rate over a plurality of training periods. The daily, short-, medium-, and long-term physiological metrics represent the average physiological metric over progressively longer periods of time. This disclosure contemplates that patterns predictive of injury risk are present in the volume, intensity, long run fraction, consistency, variability, dynamic, and physiological metrics or combinations thereof found in running-related data.

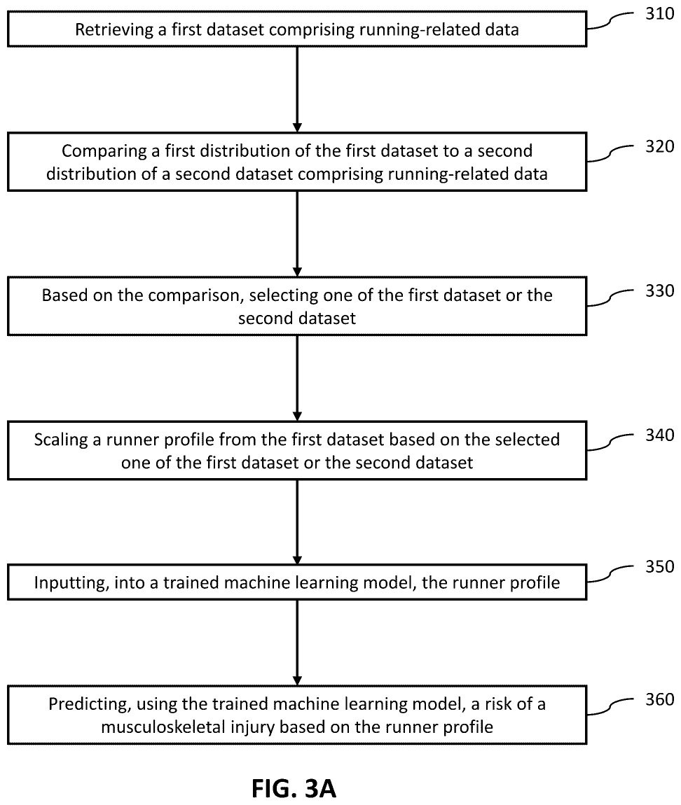

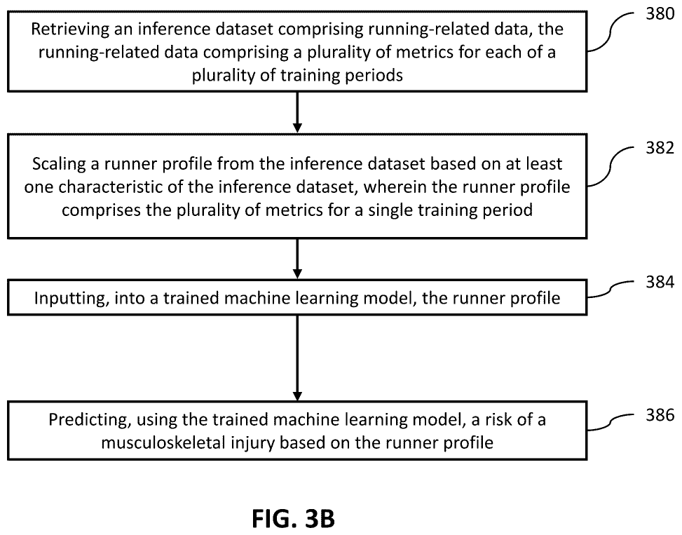

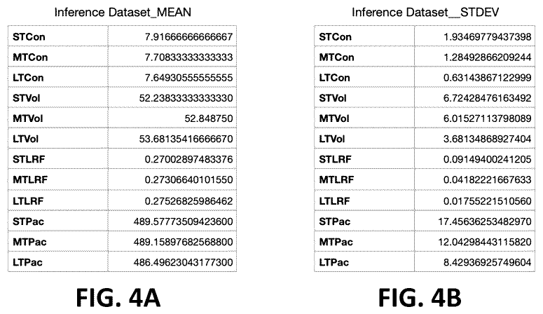

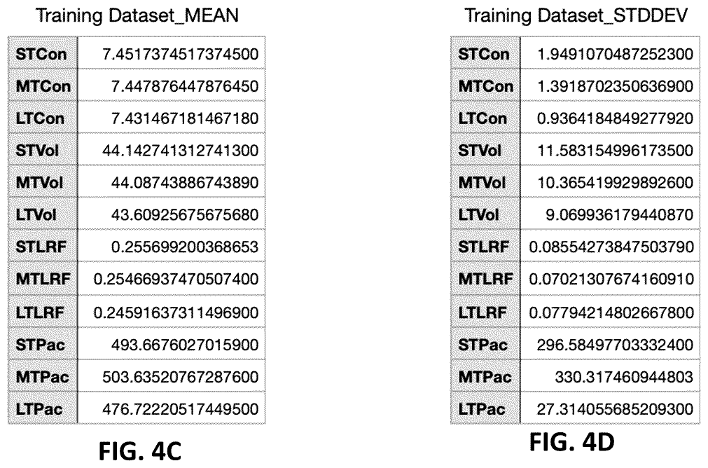

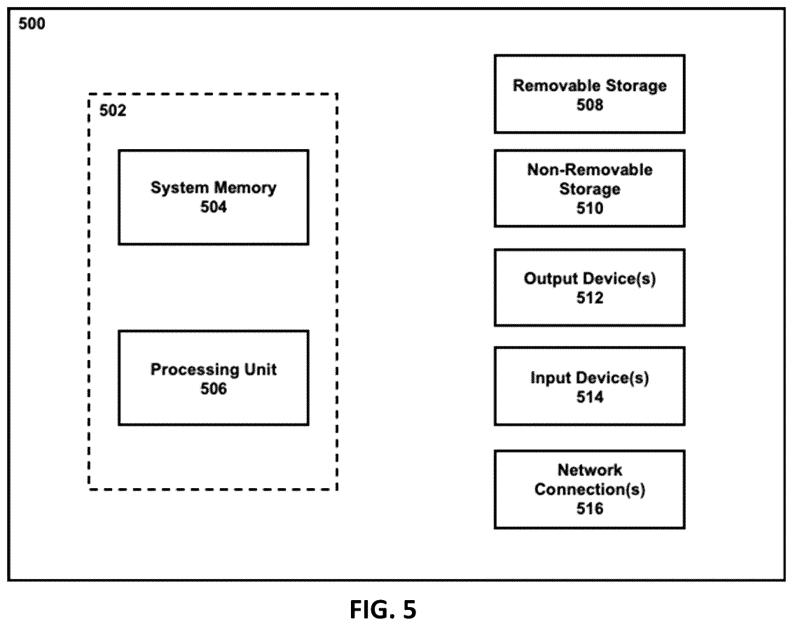

FIG. 3A is a flowchart of an example method for predicting risk of running-related injury. This disclosure contemplates that the method of FIG. 3A can be performed using one or more computing devices, e.g., computing device 500 shown in FIG. 5. At step 310, the method includes retrieving a first dataset comprising running-related data. The first dataset can optionally be stored in a data storage medium (e.g. memory). The first dataset can be grouped by week (e.g., a training period) as discussed above with regard to FIG. 2, where each row corresponds to a given week and each column corresponds to a metric. Metrics can include, but are not limited to, short-term, medium-term, and long-term metrics for each of volume, intensity, long run fraction, and consistency as shown in FIG. 2. As described herein, medium- and long-term metrics can be calculated based on the short-term metrics. In some implementations, the first dataset includes about 12 training periods of data. In some implementations, the first dataset includes about 18 training periods of data. In some implementations, the first dataset includes about 24 training periods of data. In some implementations, the first dataset includes about 30 training periods of data. In some implementations, the first dataset includes about 36 training periods of data. In the examples described herein, a training period is optionally 1 week (i.e., 7 days). Additionally, it should be understood that the number of training periods of data included in the first dataset set forth above are only provided as example. Optionally, in some implementations, the first dataset includes two or more times the number of training periods as the length of the long-term metric. For example, if the long-term metric is an average of 12 training periods (e.g., 12 weeks), then the first dataset includes at least 24 training periods (e.g., 24 weeks) of data, which is grouped by week. Optionally, the first dataset is an inference dataset. For example, in the example of FIG. 3A, the first dataset may be the example inference dataset discussed above with regard to FIGS. 4A and 4B, i.e., 40 weeks of data between the week of Oct. 31, 2022 and the week of Jul. 31, 2023. This represents the most-recent period of running-related data for the present inventor at the time of provisional application filing. Additionally, each week in the first dataset includes short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac). And as described below, the inference sample (e.g., the runner profile) can eventually be obtained from the first dataset after further processing.

The first dataset may be retrieved from an electronic runner's log and/or from a data storage medium (e.g. memory). In some implementations, the step of retrieving includes obtaining the short-term metrics (e.g. metrics for each of volume, intensity, long run fraction, and consistency) for a plurality of training periods, which are grouped by week, from the electronic runner's log and then calculating the medium- and long-term metrics as described herein. In this implementation, all of the running-related data is maintained in the electronic runner's log, which is optionally remote from the computing environment performing the operations of FIG. 3A. In other implementations, the step of retrieving includes obtaining the short-term metrics (e.g. metrics for each of volume, intensity, long run fraction, and consistency) for a plurality of training periods, which are grouped by week, from the data storage medium (e.g. memory) and then calculating the medium- and long-term metrics as described herein. In this implementation, all of the running-related data is maintained in the data storage medium (e.g. memory), which is optionally the local to or remote from the computing environment performing the operations of FIG. 3A. In yet other implementations, the step of retrieving includes obtaining the short-term metrics (e.g. metrics for each of volume, intensity, long run fraction, and consistency) for one or more training periods (e.g. recent data), which are grouped by week, from the electronic runner's log, obtaining the short-term metrics (e.g. metrics for each of volume, intensity, long run fraction, and consistency) for one or more training periods (e.g. older data), which are grouped by week, from data storage medium (e.g. memory), combining the recent and older data, and then calculating the medium- and long-term metrics as described herein. In this implementation, some of the running-related data is maintained in the electronic runner's log and some of the running-related data is maintained in the data storage medium (e.g. memory).

At step 320, the method includes comparing a first distribution of the first dataset to a second distribution of a second dataset comprising running-related data. As described below, the objective of the comparison is to determine whether a significant distribution mismatch exists. Optionally, the second dataset is a training dataset. An example training dataset is described in U.S. Pat. No. 11,515,045. For example, the training dataset can optionally include a plurality of years worth of running-related data, which is grouped by week. Similar to the first dataset, each week in the training dataset includes short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac). Additionally, the training dataset is a labeled dataset, e.g., each of the samples (i.e., a training period of data) is labeled with an injury state label (e.g., 0 for no injury and 1 for injury). In the example of FIG. 3A, the second dataset may be the example training dataset discussed above with regard to FIGS. 4C and 4D, which includes more than 5 years (i.e., between about Jan. 1, 2018 and Apr. 17, 2023) of running-related data, which is grouped by week, for the example runner (the present inventor). Prior to training a machine learning model, the training dataset can be augmented as described in U.S. Pat. No. 11,515,045.

As noted above, a first distribution of the first dataset (e.g., the example inference data) is compared to a second distribution of the second dataset (e.g., the example training data) at step 320. Comparing the respective distributions of inference and training data provides insights into any differences that might impact the performance of the trained machine learning model (i.e., the model trained with the training dataset). It is important to identify and address any significant distribution mismatches to ensure the model's generalization ability and accuracy.

In some implementations, a statistical technique is used to compare the respective distributions of the first and second datasets. For example, the statistical technique can optionally include: calculating respective summary statistics for each of the first dataset and the second dataset; and comparing the respective summary statistics of the first dataset to the respective summary statistics of the second dataset. Example summary statistics include a mean and standard deviation. FIGS. 4A and 4B are tables illustrating the mean and standard deviation of the example inference dataset, respectively. The example inference dataset includes 40 weeks (i.e. between the week of Oct. 31, 2022 and the week of Jul. 31, 2023) of running-related data, which is grouped by week, for an example runner (the present inventor). FIGS. 4C and 4D are tables illustrating the mean and standard deviation of the example training dataset, respectively. The example training dataset includes more than 5 years (i.e., between about Jan. 1, 2018 and Apr. 17, 2023) of running-related data, which is grouped by week, for the example runner (the present inventor). As shown by the FIGS. 4A-4D, the respective summary statistics differ. In other words, the distributions of the inference and training datasets are different, and these differences, if significant, can impact performance of the trained machine learning model, e.g. the model's ability to generalize to new or unseen data. It should be understood that mean and standard deviation are provided only as example summary statistics. Summary statistics can include, but are not limited to, mean, standard deviation, median, minimum, and maximum, including combinations thereof. Additionally, it should be understood that the statistical technique provided above (i.e., evaluating summary statistics) is provided only as an example. This disclosure contemplates that the statistical technique can be a statistical test that quantifies a similarity of the first dataset and the second dataset. Statistical tests include, but are not limited to, the Kolmogorov-Smirnov test or the Anderson-Darling test. The Kolmogorov-Smirnov test or the Anderson-Darling test can provide a statistical measure of the similarity or dissimilarity between the respective distributions of the first and second datasets.

As discussed above, the statistical technique can optionally include: calculating respective summary statistics for each of the first dataset and the second dataset; and comparing the respective summary statistics of the first dataset to the respective summary statistics of the second dataset, where the summary statistics include a mean and standard deviation. Example implementations are described with reference to FIGS. 4A-4D, which include mean and standard deviation for each of short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac). FIGS. 4A and 4B relate to the example inference dataset, and FIGS. 4C and 4D relate to the example training dataset.

In one implementation, the step of comparing summary statistics can include calculating a percentage difference between the respective mean values for each metric of the example inference and training datasets and determining if such percentage difference exceeds a threshold. Optionally, the threshold is about 10 percent. For example, calculate the percentage difference between respective mean values for STVol of the example inference and training datasets, i.e. the percentage difference between 52.24 and 44.14. This difference exceeds 10 percent. This calculation can be repeated for the other 11 metrics. If the percentage difference exceeds the threshold of 10 percent for greater than or equal to half of the metrics (i.e. the percentage difference for 6 or more metrics shown in FIGS. 4A-4D exceeds 10%), then a significant distribution mismatch exists.

In another implementation, the step of comparing summary statistics may include calculating a percentage difference between the respective mean values for a subset of metrics of the example inference and training datasets and determining if such percentage difference exceeds a threshold. Optionally, the subset of metrics includes only the long-term metrics. Optionally, the threshold is about 10 percent. For example, calculate the percentage difference between respective mean values for LTVol of the example inference and training datasets, i.e. the percentage difference between 53.68 and 43.61. This difference exceeds 10 percent. This calculation can be repeated for the other 3 long-term metrics. If the percentage difference exceeds the threshold of 10 percent for greater than or equal to half of the metrics (i.e. the percentage difference for 2 or more long-term metrics shown in FIGS. 4A-4D exceeds 10%), then a significant distribution mismatch exists.

Alternatively or additionally, in some implementations, a visualization technique is used to compare the respective distributions of the first and second datasets. For example, the visualization technique can include: creating respective histograms for each of the first dataset and the second dataset; plotting the respective histograms for each of the first dataset and the second dataset; and comparing the respective histogram for the first dataset to the respective histogram for the second dataset. This disclosure contemplates that a visualization such as respective histograms can be used to understand whether the distributions of the first and second datasets are different, which may impact performance of the trained machine learning model. It should be understood that a histogram is provided only as an example plot for visualizing dataset distribution. Alternative plots for visualizing dataset distribution can include, but are not limited to, Kernel density estimation (KDE) plot and Quantile-Quantile (Q-Q) plot. A KDE plot can provide a smooth estimate of the probability density function (PDF) and can help visualize the shape and overlap of the respective distributions of the first and second datasets. A Q-Q plot can be used to assess how well the distributions match by comparing the quantiles of the two the first and second datasets. If the points lie approximately on a straight line, it suggests that the distributions are similar.

At step 330, the method includes selecting one of the first dataset or the second dataset based on the comparison. As described above, a threshold can be set by the user to identify significant distribution mismatches between the first dataset (e.g., the example inference dataset) and the second dataset (e.g., the example training dataset). As described herein, inference data is typically scaled in the same manner as the training data. This is good practice assuming that the training data is representative of the distribution of the inference data. In cases, however, where the training data is not representative of the distribution of the inference data (i.e., there is a significant distribution mismatch), the inference data can be scaled in a different manner, for example using the mean and standard deviation of the inference dataset (instead of using the mean and standard deviation of the training dataset). Scaling the inference data based on at least one characteristic of the inference dataset may have advantages where the trained model is configured to predict an anomaly such as a running-related injury. Accordingly, step 330 results in selection of a dataset (e.g., training or inference) to which a runner profile from the inference dataset is to be scaled. In cases with insignificant distribution mismatches between the inference dataset and the training dataset, the runner profile from the inference dataset can be scaled based on one or more characteristics of the training dataset. This would be typical for machine learning practice. On the other hand, in cases with significant distribution mismatches between the inference dataset and the training dataset, the runner profile from the inference dataset can be scaled based on one or more characteristics of the inference dataset. This would not be typical for machine learning practice. Such scaling practice improves the trained model's ability to generalize to new or unseen data.

At step 340, the method includes scaling a runner profile from the first dataset based on the selected one of the first dataset or the second dataset. For example, the first dataset (e.g., the example inference dataset) can be selected when there is a significant distribution mismatch between it and the second dataset (e.g., the example training dataset) at step 330. Thus, instead of scaling the runner profile from the first dataset in the same manner as scaling used for the second dataset, which would be typical for machine learning practice, the runner profile from the first dataset can be scaled based on one or more characteristics of the first dataset. As used herein, a scaled runner profile or dataset has undergone a scaling process, where the values of the data have been transformed to a specific range or distribution. Scaling is a preprocessing step in machine learning to ensure that all features have a similar scale and to prevent certain features from dominating the learning process due to their larger values. Two example scaling processes are standardization (also known as z-score normalization) and normalization (also known as min-max scaling). This disclosure contemplates performing data scaling using tools known in the art including, but not limited to using a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit. It should be understood that standardization and normalization are provided only as example scaling techniques. This disclosure contemplates using other scaling techniques with the implementations described herein.

In some implementations, the runner profile from the first dataset is scaled by standardizing the runner profile from the first dataset based on at least one characteristic of the first dataset. Standardization (also known as z-score normalization or z-score scaling) transforms the data of the first dataset to have a mean of zero and a standard deviation of one. For example, standardization is performed using Equation (1) below, resulting in a distribution centered around zero with a unit variance. Standardization is useful because the features in running-related data have different scales and units and therefore it helps to bring them to a common scale.

$${X}_{standardized} = \frac{χ - μ}{σ} \qquad ( 1 )$$

where X_{standardized} is the standardized value of the data point x, x is the original value of the data point, μ is the mean of the dataset, and σ is the standard deviation of the dataset. According to Equation (1) the mean (u) is subtracted from each data point (x) and then divided by the standard deviation (σ). This transformation centers the data around zero and scales it based on the spread of the data.

In some implementations, the runner profile from the first dataset is scaled by normalizing the runner profile from the first dataset based on at least one characteristic of the first dataset. Normalization (also known as min-max scaling) scales the data of the first dataset to a fixed range, typically between 0 and 1. For example, normalization is performed using Equation (2) below. Normalization is useful when it is desired to preserve the relative relationships and proportions between the data points.

$${X}_{normalized} = \frac{χ - {χ}^{min}}{{χ}^{max} - {χ}^{min}} \qquad ( 2 )$$

where X_{normalized} is the normalized value of the data point x, x is the original value of the data point, χmin is the minimum value in the dataset, and χmax is the maximum value in the dataset. According to Equation (2) the minimum value (xₘᵢₙ) is subtracted from each data point (x) and then divided by the range (xₘₐₓ−Xₘᵢₙ). This process scales the data to a range between 0 and 1, where the minimum value becomes 0 and the maximum value becomes 1.

Optionally, prior to scaling at step 340, the runner profile is extracted from the first dataset. The runner profile includes metrics for a single training period. As described herein, the runner profile may be associated with the most-recent training period (e.g. week) for a retrospective analysis or associated with a future training period (e.g. week) for a prospective analysis. In some implementations, the runner profile includes at least one volume metric, at least one intensity metric, and at least one long run fraction metric. Optionally, the runner profile further includes one or more of at least one consistency metric, at least one variability metric, at least one dynamic metric, or at least one physiological metric. For example, in some implementations, the runner profile includes at least one consistency metric, at least one volume metric, at least one intensity metric, and at least one long run fraction metric. Such an implementation is described in the Examples below. Additionally, as described below, the runner profile is a scaled runner profile or feature vector.

An example unscaled runner profile and scaled runner profile (also referred to herein as “feature vector”) extracted from the data associated with the week of Jul. 31, 2023 in the example inference dataset is provided below. The feature vector has been standardized based on characteristics of the example inference dataset, e.g. using the mean and standard deviation shown in FIGS. 4A and 4B, respectively. The unscaled/scaled runner profile includes (in order) short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac).

Unscaled Runner Profile

| | | | |
|---|---|---|---|
| [[1.40000000e+01 | 1.16666667e+01 | 8.75000000e+00 | 4.60200000e+01 |
| 4.96233333e+01 | 5.03641667e+01 | 1.95784442e−01 | 2.24654677e−01 |
| 2.44558346e−01 | 4.81551499e+02 | 4.87855176e+02 | 4.98658107e+02]] |

Feature Vector (Scaled Runner Profile)

| | | | |
|---|---|---|---|
| [3.14433259 | 3.08058607 | 1.74315336 | −0.92475758 |
| −0.5362047 | −0.90107941 | −0.81146885 | −1.15755997 |
| −1.74963181 | −0.45978856 | −0.1082623 | 1.44279791] |

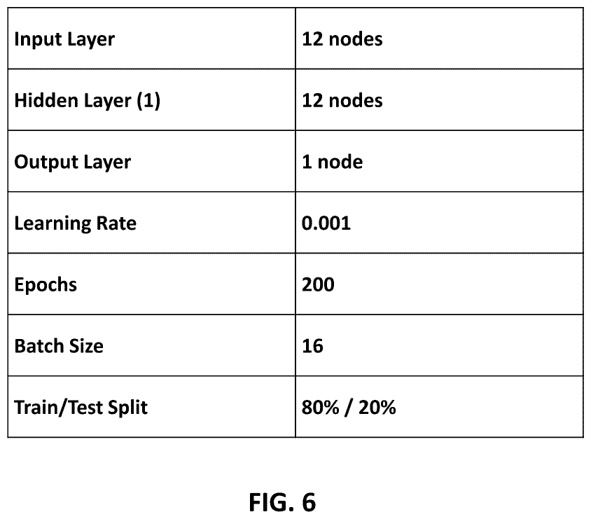

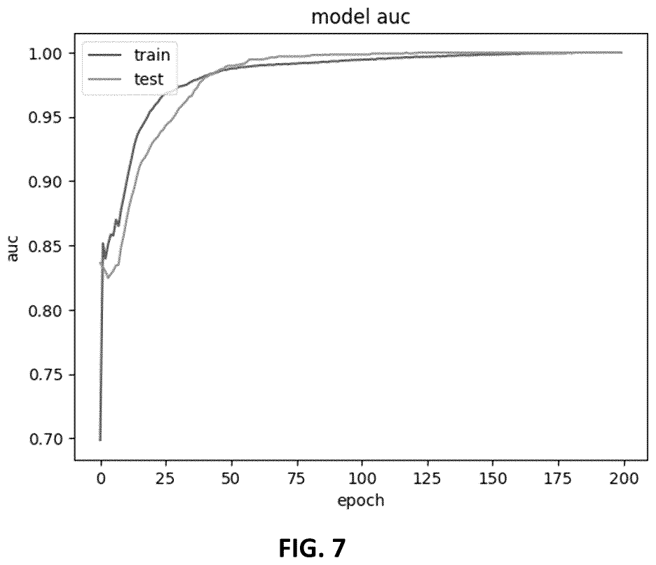

At step 350, the method includes inputting, into a trained machine learning model, the runner profile. The runner profile is a “feature vector”, e.g. the feature vector shown above. In other words, the runner profile input into the trained machine learning model has been scaled. This disclosure contemplates that the trained machine learning model can be the machine learning model 100 shown in FIG. 1 such that the runner profile is the input 110 to the machine learning model 100 of FIG. 1. The runner profile input into the trained machine learning model is a vector or tensor (see feature vector above). In some implementations, the trained machine learning model is a deep learning model. Alternatively or additionally, in some implementations, the trained machine learning model is an artificial neural network. Optionally, the trained machine learning model can be the example ANN described in the Example below (see FIGS. 6 and 7). It should be understood that the trained ANN described in the Examples is provided only as an example. This disclosure contemplates using other trained machine learning models with the techniques described herein.

At step 360, the method includes predicting, using the trained machine learning model, a risk of a musculoskeletal injury based on the runner profile. This disclosure contemplates that the trained machine learning model can be the machine learning model 100 shown in FIG. 1 such that the risk of musculoskeletal injury is the output 120 of the machine learning model 100 of FIG. 1. As described herein, the trained machine learning model is configured to analyze input “features” and predict risk of musculoskeletal injury based on the same. In some implementations, the trained machine learning model outputs a probability of musculoskeletal injury (e.g., a logistic regression). Alternatively, the trained machine learning model classifies the runner profile into a plurality of risk categories (e.g., logistic regression classification). Risk categories can optionally include injury/no injury, low risk/high risk, low risk/medium risk/high risk, etc. classifications. As described herein, the musculoskeletal injury is a running-related injury such as an injury affecting the runner's bones, joints, or soft tissue.

In some implementations, the runner profile input into the model at step 350 includes metrics from the most-recent training period. In other implementations, the runner profile input into the model at step 350 includes metrics calculated for the next (e.g., future) training period. A prospective runner profile can be calculated, for example, based on the runner's training plan (volume, intensity, etc.) for an upcoming training period. In either implementation, the prediction at step 360 allows the runner to assess, adjust, tailor, etc. his training schedule to minimize likelihood of, or in some cases avoid, suffering a musculoskeletal injury.

Optionally, in some implementations, the method includes adjusting a training plan for a runner based on the prediction of step 360. This may include one or more of: reducing the number of planned runs in the next training period, reducing the planned volume in the next training period, reducing the planned intensity in the next training period, and/or reducing the planned long run volume in the next training period. Optionally, the method includes performing the method of FIG. 3A after the adjustment. In other words, this disclosure contemplates an iterative process to identify a training plan that minimizes the likelihood of musculoskeletal injury.

Referring now to FIG. 3B, a flowchart of another example method for predicting risk of running-related injury is shown. This disclosure contemplates that the method of FIG. 3B can be performed using one or more computing devices, e.g., computing device 500 shown in FIG. 5. At step 380, the method includes retrieving an inference dataset comprising running-related data. The inference dataset may be retrieved from an electronic runner's log and/or from a data storage medium (e.g. memory). Dataset retrieval is described above with regard to FIG. 3A. The inference dataset can optionally be stored in a data storage medium (e.g. memory).

The inference dataset includes running-related data for a plurality of training periods (e.g. weeks). The running-related data includes a plurality of metrics for each of a plurality of training periods. In other words, the inference dataset is not limited to data for a single training period. It instead includes data for a plurality of training periods. In fact, it is insufficient to retrieve data for a single training period for the method of FIG. 3B at least because of the data scaling technique (e.g. described below at step 382), which requires information about one or more characteristics of the inference dataset. In some implementations, the inference dataset includes running-related data, which is grouped by week, for at least two times the number of training periods averaged for a long-term metric. For example, if the long-term metric is an average of 12 training periods, then the inference dataset includes running-related data, which is grouped by week, for at least 24 training periods. In some implementations, the inference dataset includes running-related data, which is grouped by week, for between two and four times the number of training periods averaged for a long-term metric. For example, if the long-term metric is an average of 12 training periods, then the inference dataset includes running-related data, which is grouped by week, for between 24 and 48 training periods. Optionally, the inference dataset includes running-related data, which is grouped by week, for about three times the number of training periods averaged for a long-term metric. For example, if the long-term metric is an average of 12 training periods, then the inference dataset includes running-related data, which is grouped by week, for about 36 training periods. An example inference dataset is discussed above. It includes 40 weeks (i.e. between the week of Oct. 31, 2022 and the week of Jul. 31, 2023) of running-related data, which is grouped by week, for an example runner (the present inventor).

At step 382, the method includes scaling a runner profile from the inference dataset based on at least one characteristic of the inference dataset. Thus, instead of scaling the runner profile from the inference dataset in the same manner used for scaling the dataset used for training (i.e. a training dataset) a machine learning model, which would be typical for machine learning practice, the runner profile can be scaled based on one or more characteristics (e.g. mean, standard deviation, minimum value, maximum value, etc.) of the inference dataset. The inference dataset is different than the training dataset. For example, if the inference and training datasets are associated with the same runner, the inference and training datasets may cover different periods of time. Or the inference and training datasets may be associated with different runners. In either case, one may expect distributions of the inference and training datasets to be different.

In some implementations, the runner profile from the inference dataset is scaled by standardizing the runner profile based on one or more characteristics of the inference dataset. Standardization (also known as z-score normalization or z-score scaling) transforms the data of the runner profile from the inference dataset to have a mean of zero and a standard deviation of one. For example, standardization is performed using Equation (1) above. In some implementations, the runner profile from the inference dataset is scaled by normalizing the runner profile based on one or more characteristics of the inference dataset. Normalization (also known as min-max scaling) scales the data of the inference dataset to a fixed range, typically between 0 and 1. For example, normalization is performed using Equation (2) above.

Optionally, prior to scaling at step 382, the runner profile is extracted from the inference dataset. As described herein, the runner profile may be associated with the most-recent training period (e.g. week) for a retrospective analysis or associated with a future training period (e.g. week) for a prospective analysis. The runner profile includes the plurality of metrics for a single training period. In some implementations, the runner profile includes at least one volume metric, at least one intensity metric, and at least one long run fraction metric. Optionally, the runner profile further includes one or more of at least one consistency metric, at least one variability metric, at least one dynamic metric, or at least one physiological metric. For example, in some implementations, the runner profile includes at least one consistency metric, at least one volume metric, at least one intensity metric, and at least one long run fraction metric. Such an implementation is described in the Examples below.

An example unscaled runner profile and scaled runner profile (also referred to herein as “feature vector”) extracted from the data associated with the week of Jul. 31, 2023 in the example inference dataset is provided below. The feature vector has been standardized based on the characteristics of the example inference dataset, e.g. using the mean and standard deviation shown in FIGS. 4A and 4B, respectively. The unscaled/scaled runner profile includes (in order) short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac).

Unscaled Runner Profile

| | | | |
|---|---|---|---|
| [[1.40000000e+01 | 1.16666667e+01 | 8.75000000e+00 | 4.60200000e+01 |
| 4.96233333e+01 | 5.03641667e+01 | 1.95784442e−01 | 2.24654677e−01 |
| 2.44558346e−01 | 4.81551499e+02 | 4.87855176e+02 | 4.98658107e+02]] |

Feature Vector (Scaled Runner Profile)

| | | | |
|---|---|---|---|
| [3.14433259 | 3.08058607 | 1.74315336 | −0.92475758 |
| −0.5362047 | −0.90107941 | −0.81146885 | −1.15755997 |
| −1.74963181 | −0.45978856 | −0.1082623 | 1.44279791] |

At step 384, the method includes inputting, into a trained machine learning model, the runner profile. The runner profile is a “feature vector”, e.g. the feature vector shown above. This disclosure contemplates that the trained machine learning model can be the machine learning model 100 shown in FIG. 1 such that the runner profile is the input 110 to the machine learning model 100 of FIG. 1. The runner profile input into the trained machine learning model is a vector or tensor (see feature vector above). In some implementations, the trained machine learning model is a deep learning model. Alternatively or additionally, in some implementations, the trained machine learning model is an artificial neural network. Optionally, the trained machine learning model can be the example ANN described in the Example below (see FIGS. 6 and 7). It should be understood that the trained ANN described in the Example is provided only as an example. This disclosure contemplates using other trained machine learning models with the techniques described herein.

At step 386, the method includes predicting, using the trained machine learning model, a risk of a musculoskeletal injury based on the runner profile. This disclosure contemplates that the trained machine learning model can be the machine learning model 100 shown in FIG. 1 such that the risk of musculoskeletal injury is the output 120 of the machine learning model 100 of FIG. 1. As described herein, the trained machine learning model is configured to analyze input “features” and predict risk of musculoskeletal injury based on the same. In some implementations, the trained machine learning model outputs a probability of musculoskeletal injury (e.g., a logistic regression). Alternatively, the trained machine learning model classifies the runner profile into a plurality of risk categories (e.g., logistic regression classification). Risk categories can optionally include injury/no injury, low risk/high risk, low risk/medium risk/high risk, etc. classifications. As described herein, the musculoskeletal injury is a running-related injury such as an injury affecting the runner's bones, joints, or soft tissue.

In some implementations, the runner profile input into the model at step 384 includes metrics from the most-recent training period. In other implementations, the runner profile input into the model at step 384 includes metrics calculated for the next (e.g., future) training period. A prospective runner profile can be calculated, for example, based on the runner's training plan (volume, intensity, etc.) for an upcoming training period. In either implementation, the prediction at step 386 allows the runner to assess, adjust, tailor, etc. his training schedule to minimize likelihood of, or in some cases avoid, suffering a musculoskeletal injury.

Optionally, in some implementations, the method includes adjusting a training plan for a runner based on the prediction of step 386. This may include one or more of: reducing the number of planned runs in the next training period, reducing the planned volume in the next training period, reducing the planned intensity in the next training period, and/or reducing the planned long run volume in the next training period. Optionally, the method includes performing the method of FIG. 3A after the adjustment. In other words, this disclosure contemplates an iterative process to identify a training plan that minimizes the likelihood of musculoskeletal injury.

It should be appreciated that the logical operations described herein with respect to the various figures may be implemented (1) as a sequence of computer implemented acts or program modules (i.e., software) running on a computing device (e.g., the computing device described in FIG. 5), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 5, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 5 by box 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, values, etc.), but some errors and deviations should be accounted for.

### Example 1

In Example 1, a labeled dataset including running-related data for one individual runner is created. The individual runner is the inventor of the present application. The labeled dataset was collected and used to demonstrate the feasibility of applying machine learning to predict risk of running-related injury. The labeled dataset contains the individual's running-related data downloaded from the GARMIN CONNECT website of Garmin International of Olathe, Kansas in XLS file format. It should be understood that XLS format is only an example and that data may be downloaded in other file formats including, but not limited to, CSV file format. In addition to data downloaded from the GARMIN CONNECT website, each sample was tagged with a label-0 for non-injury state and 1 for injury state. Thus, the dataset is a labeled dataset. The dataset includes running-related data that is grouped by week for the period between Jan. 1, 2018 and Apr. 17, 2023. Each week (i.e., sample) is tagged with an injury/non-injury label. A labeled sample thus includes a plurality of metrics and corresponding label associated with a given week.

The 3 samples tagged as injury class are the weeks of Aug. 31, 2020; Apr. 22, 2019; and Jul. 2, 2018, which represent the weeks of injury occurrence. The Aug. 31, 2020 injury was to the right calf (possible soleus muscle strain) and resulted in the individual runner taking 4 consecutive days off (i.e., no running) during the following week of Sep. 7, 2020. The Apr. 22, 2019 injury was to the right knee (possible patellar tendonitis) and resulted in the individual runner taking a substantial amount of time (i.e., no running) off during the following eight weeks. The Jul. 2, 2018 injury was to the right groin (possible groin strain) and resulted in the individual runner taking 4 consecutive days off (i.e., no running) spanning the weeks of July 2 and 9, 2018.

The labeled dataset is unbalanced because samples in the injury class are underrepresented. For example, there are only 3 samples tagged with the injury state label (1), while more than 250 samples are tagged with the non-injury state label (0). Therefore, the labeled dataset was augmented as described in U.S. Pat. No. 11,515,045. In particular, a plurality of synthetic samples were created based on 3 samples tagged as injury class (i.e., the samples for weeks of Aug. 31, 2020; Apr. 22, 2019; and Jul. 2, 2018), and such synthetic samples were appended to the labeled dataset.

After augmenting the dataset, the metrics were scaled. Scaling was accomplished using the PANDAS tool kit in the Python programming language. Both the Python programming language and the PANDAS tool kit, which is a data analysis tool, are well known in the art and therefore not described herein. In the example, the augmented dataset was standardized (see e.g., Equation (1)).

The scaled, augmented dataset was used to train a machine learning model. Various ANNs were trained using the scaled, augmented dataset. Model training was accomplished using the KERAS tool kit in the Python programming language. Both the Python programming language and the KERAS tool kit, which is a deep learning framework, are well known in the art and therefore not described herein. In particular, the scaled, augmented dataset was read into a data frame using the Python programming language. In the example, 80% of the scaled, augmented dataset serves as the training dataset and 20% of the scaled, augmented dataset serves as the testing dataset. Train/test splitting of the scaled, augmented dataset and model training is accomplished using functions in the KERAS tool kit. This includes selecting model architecture and hyperparameter.

Various ANNs were trained using the scaled, augmented dataset and evaluated for their ability to distinguish between injury/non-injury classes using area under the receiver operator curve (AUC) as the evaluation metric. AUC provides an aggregate measure of performance across all possible classification thresholds, i.e., a measure of the model's ability to distinguish between the injured state and non-injured state classes. Higher AUC is associated with better performance.

In the example, the following 12 metrics serve as model features: short-, medium-, and long-term consistency metrics; short-, medium-, and long-term volume metrics; short-, medium-, and long-term long run fraction metrics; and short-, medium-, and long-term long run intensity metrics. The model target is the Injury Label. ANNs with different architectures were tested, including ANNs with 1 input layer, 1 hidden layer or 2 hidden layers, and 1 output layer. As one example, an ANN with 1 input layer (12 nodes), 1 hidden layer (12 nodes), and 1 output layer (1 node) is referenced in FIG. 6. As shown in FIG. 7, the trained ANN of FIG. 6 performed very well with AUC equal to 1.

The trained ANN of FIG. 6 was saved to a file in a hierarchal data format (HDF) file format. The ANN is configured to distinguish between injured state and non-injured state classes based on the following features: short-, medium-, and long-term consistency metrics; short-, medium-, and long-term volume metrics; short-, medium-, and long-term long run fraction metrics; and short-, medium-, and long-term long run intensity metrics. Hyperparameters for the trained ANN include learning rate=0.001, epochs=200, and batch size=16 as shown in FIG. 6. It should be understood that hyperparameters may be optimized to improve performance, which was not necessary for the trained ANN.

The trained ANN of FIG. 6 has been deployed by the individual runner in inference mode beginning around May 1, 2023. Deployment of previous versions of the ANN are described in U.S. Pat. No. 11,515,045. Model deployment was accomplished using the PANDAS, NUMPY, and KERAS tool kits in the Python programming language, which are all well known in the art. In particular, the trained ANN (i.e., HDF file format) and a runner profile were uploaded using the Python programming language. During deployment, a runner profile is input into the trained ANN of FIG. 6. The runner profile is a feature vector. An example feature vector for week of Jul. 31, 2023 is provided below.

Feature Vector (Scaled Runner Profile) for Week of Jul. 31, 2023

| | | | |
|---|---|---|---|
| [3.14433259 | 3.08058607 | 1.74315336 | −0.92475758 |
| −0.5362047 | −0.90107941 | −0.81146885 | −1.15755997 |
| −1.74963181 | −0.45978856 | −0.1082623 | 1.44279791] |

The runner profile (i.e. feature vector above) includes (in order) the following features: short-, medium-, and long-term consistency metrics (3.14433259, 3, 08058607, 1.74315336); short-, medium-, and long-term volume metrics (−0.92475758, −0.5362047, −0.90107941); short-, medium-, and long-term long run fraction metrics (−0.81146885, −1.15755997, −1.74963181); and short-, medium-, and long-term long run intensity metrics (−0.45978856, −0.1082623, 1.44279791). For the week of Jul. 31, 2023, the short-term metrics are retrospective, i.e., based on the individual runner's completed training plan for a past week. In other words, the respective values for the short term metrics are based on the individual runner's actual metrics for a completed week. In other examples, the short-term metrics can be prospective, i.e., based on the individual runner's training plan for a future week. In other words, the respective values for the short term metrics can be based on the individual runner's expectations for a future week (e.g. the next week). The trained ANN of FIG. 6 outputs a prediction based on the input runner profile.

### Example 2

In Example 2, the present inventor's runner profile associated with the week of Jan. 8, 2024 of an inference dataset is scaled according to two different techniques: (A) scaling based on the mean and standard deviation of the inference dataset and (B) scaling based on the mean and standard deviation of the example training dataset shown in FIGS. 4C and 4D, respectively. The scaled runner profile (feature vector) is then input into the trained ANN of FIG. 6. As shown below, the prediction output from the trained ANN of FIG. 6 is different. In particular, the predicted risk was LOW for scaling technique (A) where the profile was scaled based on characteristics of the inference dataset, while predicted risk was HIGH for scaling technique (B) where the profile was scaled based on characteristics of the training dataset. In other words, different scaling techniques resulted in different predictions. The present inventor did not sustain an injury during the week of Jan. 8, 2024, so the scaling technique (A) prediction was consistent with outcome. Example 2 demonstrates that the scaling technique may affect the the prediction. In Example 2, the trained ANN of FIG. 6 was better able to generalize to the new data (i.e. feature vector from week of Jan. 8, 2024) when scaling technique (A) was employed (because of differences between inference and training datasets).

Scaling Technique (A): The runner profile was standardized based on characteristics of the inference dataset, e.g. using the mean and standard deviation of the inference dataset. The unscaled/scaled runner profile includes (in order) short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac).

Unscaled Runner Profile

| | | | |
|---|---|---|---|
| [[8.00000000e+00 | 7.33333333e+00 | 7.91666667e+00 | 5.30000000e+01 |
| 4.41566667e+01 | 4.79458333e+01 | 2.45283019e−01 | 2.34488348e−01 |
| 2.50394805e−01 | 4.83018868e+02 | 4.89318336e+02 | 4.90875119e+02]] |

Feature Vector (Scaled Runner Profile)

| | | | |
|---|---|---|---|
| [−0.02704679 | −0.78072113 | −1.17342243 | 0.64217382 |
| −1.52315182 | −2.46246612 | 0.05730261 | −0.72548087 |
| 1.48988674 | −0.61823329 | −0.33571956 | −0.44240896] |

Prediction
Your injury risk is LOW

Scaling Technique (B): The runner profile was standardized based on characteristics of the training dataset, e.g. using the mean and standard deviation shown in FIGS. 4C and 4C, respectively. The unscaled/scaled runner profile includes (in order) short-term, medium-term, and long-term metrics for consistency (STCon, MTCon, LTCon), volume (STVol, MTVol, LTVol), long run fraction (STLrf, MTLrf, LTLrf), and intensity (STPac, MTPac, LTPac).

Unscaled Runner Profile

| | | | |
|---|---|---|---|
| [[8.00000000e+00 | 7.33333333e+00 | 7.91666667e+00 | 5.30000000e+01 |
| 4.41566667e+01 | 4.79458333e+01 | 2.45283019e−01 | 2.34488348e−01 |
| 2.50394805e−01 | 4.83018868e+02 | 4.89318336e+02 | 4.90875119e+02]] |

Feature Vector (Scaled Runner Profile)

| | | | |
|---|---|---|---|
| [0.28128909 | −0.08229439 | 0.51814386 | 0.7646672 |
| 0.00667873 | 0.47812647 | −0.12176582 | −0.28742547 |
| 0.05745841 | −0.0359045 | −0.04334276 | 0.51815499] |

Prediction
Your injury risk is HIGH. Be careful!

### Example 3

In Example 3, the present inventor used the prediction output from the trained ANN of FIG. 6 to guide his training. For example, on Dec. 31, 2023, the inventor initially planned the following for the upcoming week of Jan. 1, 2024:8 total runs (includes 6 running days with 1 workout (where this day includes 3 separate runs-warmup, workout session, cooldown) and 1 day off), 53.5 total miles, a 13.5 mile long run, and an average pace of about 8:01 minutes per mile. The short-term metrics were therefore as follows: STCon=8, STVol=53.5, STLrf=0.2523, STPac=481. An inference dataset of the present inventor's running-related data for the previous 36 weeks plus the plan for the future week of Jan. 1, 2024 was created. The present inventor's runner profile associated with the week of Jan. 1, 2024 (i.e. a single training period) was scaled based on the mean and standard deviation of the inference dataset. The scaled runner profile (feature vector) was then input into the trained ANN of FIG. 6. As shown below, the prediction output from the trained ANN of FIG. 6 was HIGH.

Unscaled Runner Profile

| | | | |
|---|---|---|---|
| [[8.00000000e+00 | 7.00000000e+00 | 7.91666667e+00 | 5.35000000e+01 |
| 4.09033333e+01 | 4.87308333e+01 | 2.52336449e−01 | 2.52298349e−01 |
| 2.51142637e−01 | 4.81308411e+02 | 4.93545758e+02 | 4.90466337e+02]] |

Feature Vector (Scaled Runner Profile)

| | | | |
|---|---|---|---|
| [−0.10101525 | −1.16073485 | −1.22104437 | 0.71023988 |
| −2.9275094 | −2.15387799 | 0.2812331 | 0.63860507 |
| 1.63450496 | −0.77235869 | 0.27702792 | −0.60609061] |

Prediction
Your injury risk is HIGH. Be careful!

Since the predicted risk was HIGH, the present inventor adjusted his plan for the upcoming week of Jan. 1, 2024:8 total runs (includes 6 running days with 1 workout (where this day includes 3 separate runs-warmup, workout session, cooldown) and 1 day off), 51 total miles, a 12 mile long run, and an average pace of about 8:05 minutes per mile. The short-term metrics were therefore as follows: STCon=8, STVol=51, STLrf=0.2353, STPac=485. In other words, the planned adjustments included slight changes to total volume (−2.5 miles), long run distance (−1.5 miles), and pace (˜4 seconds slower per mile) as compared to the initial plan. An inference dataset of the present inventor's running-related data for the previous 36 weeks plus the adjusted plan for the future week of Jan. 1, 2024 was created. The present inventor's runner profile associated with the week of Jan. 1, 2024 (i.e. a single training period) was scaled based on the mean and standard deviation of the inference dataset. The scaled runner profile was then input into the trained ANN of FIG. 6. As shown below, the prediction output from the trained ANN of FIG. 6 was LOW.

Unscaled Runner Profile

| | | | |
|---|---|---|---|
| [[8.00000000e+00 | 7.00000000e+00 | 7.91666667e+00 | 5.10000000e+01 |
| 4.00700000e+01 | 4.85225000e+01 | 2.35294118e−01 | 2.46617572e−01 |
| 2.49722443e−01 | 4.85294118e+02 | 4.95491224e+02 | 4.90854758e+02]] |

Feature Vector (Scaled Runner Profile)

| | | | |
|---|---|---|---|
| [−0.10101525 | −1.16073485 | −1.22104437 | 0.29830778 |
| −3.08695893 | −2.35558612 | −0.27427537 | 0.20263335 |
| 1.40925142 | −0,50068181 | 0.56978048 | −0.51637347] |

Prediction
Your injury risk is LOW.

The present inventor executed according to the adjusted plan and did not sustain an injury during the week of Jan. 1, 2024. This example demonstrates how a trained ANN can be deployed to assist a runner in avoiding injuries by adjusting training plan for future training periods.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for predicting risk of running-related injury, the method comprising:
retrieving a first dataset comprising running-related data;
comparing a first distribution of the first dataset to a second distribution of a second dataset comprising running-related data;
based on the comparison, selecting one of the first dataset or the second dataset;
scaling a runner profile from the first dataset based on the selected one of the first dataset or the second dataset;
inputting, into a trained machine learning model, the runner profile; and
predicting, using the trained machine learning model, a risk of a musculoskeletal injury based on the runner profile.

2. The method of claim 1, wherein comparing the first distribution of the first dataset to the second distribution of the second dataset comprises using a statistical technique.

3. The method of claim 2, wherein the statistical technique comprises:
calculating respective summary statistics for each of the first dataset and the second dataset; and
comparing the respective summary statistics of the first dataset to the respective summary statistics of the second dataset.

4. (canceled)

5. The method of claim 1, wherein comparing the first distribution of the first dataset to the second distribution of the second dataset comprises using a visualization technique.

6. The method of claim 5, wherein the visualization technique comprises:
creating respective histograms for each of the first dataset and the second dataset;
plotting the respective histograms for each of the first dataset and the second dataset; and
comparing the respective histogram for the first dataset to the respective histogram of the second dataset.

7. The method of claim 1, wherein scaling the runner profile from the first dataset based on the selected one of the first dataset or the second dataset comprises standardizing or normalizing the runner profile from the first dataset based on at least one characteristic of the selected one of the first dataset or the second dataset.

8. (canceled)

9. The method of claim 1, wherein the first dataset is an inference dataset, and the second dataset is a training dataset, and wherein the selected one of the first dataset or the second dataset is the inference dataset.

10. (canceled)

11. The method of claim 1, wherein the first dataset and the second dataset comprise running-related data for a same runner, or wherein each of the first dataset and the second dataset comprises running-related data for a different runner.

12. (canceled)

13. The method of claim 1, wherein the runner profile comprises at least one volume metric, at least one intensity metric, and at least one long run fraction metric.

14. (canceled)

15. (canceled)

16. (canceled)

17. The method of claim 13, wherein the runner profile further comprises one or more of at least one consistency metric, at least one variability metric, at least one dynamic metric, or at least one physiological metric.

18. The method of claim 1, wherein the trained machine learning model is configured to predict the risk of the musculoskeletal injury by classifying the runner profile into one of a plurality of risk categories, or wherein the trained machine learning model is configured to predict the risk of the musculoskeletal injury by providing a probability of the musculoskeletal injury.

19. (canceled)

20. (canceled)

21. (canceled)

22. The method of claim 1, further comprising adjusting a training plan based on the predicted risk of the musculoskeletal injury.

23. (canceled)

24. A method for predicting risk of running-related injury, the method comprising:
retrieving an inference dataset comprising running-related data, the running-related data comprising a plurality of metrics for each of a plurality of training periods;
scaling a runner profile from the inference dataset based on at least one characteristic of the inference dataset, wherein the runner profile comprises the plurality of metrics for a single training period;
inputting, into a trained machine learning model, the runner profile; and
predicting, using the trained machine learning model, a risk of a musculoskeletal injury based on the runner profile.

25. The method of claim 24, wherein a number of the plurality of training periods is at least two times greater than a number of training periods averaged for a long-term metric.

26. The method of claim 24 or 25, wherein the trained machine learning model is trained using a training dataset, wherein the training dataset is different than the inference dataset.

27. The method of claim 26, wherein the inference dataset and the training dataset comprise running-related data for a same runner, or wherein each of the inference dataset and the training dataset comprises running-related data for a different runner.

28. (canceled)

29. The method of claim 24, wherein the at least one characteristic of the inference dataset comprises a mean or a standard deviation.

30. The method of claim 24, wherein the plurality of metrics comprises at least one volume metric, at least one intensity metric, and at least one long run fraction metric.

31. The method of claim 24, further comprising adjusting a training plan based on the predicted risk of the musculoskeletal injury.

32. A system for predicting risk of running-related injury, the system comprising:
at least one processor and at least one memory, the at least one memory having computer-executable instructions stored thereon that, when executed by the at least one processor, cause the at least one processor to:
retrieve an inference dataset comprising running-related data, the running-related data comprising a plurality of metrics for each of a plurality of training periods;
scale a runner profile from the inference dataset based on at least one characteristic of the inference dataset, wherein the runner profile comprises the plurality of metrics for a single training period;
input, into a trained machine learning model, the runner profile; and
predict, using the trained machine learning model, a risk of a musculoskeletal injury based on the runner profile.
